(19) United States
(12) Patent Application Publication
(10) Pub. No.: US 2025/0131670 A1
(43) Pub. Date: Apr. 24, 2025

(54) IMAGE PROCESSING APPARATUS, IMAGE PROCESSING METHOD, AND RECORDING MEDIUM

(71) Applicant: LUXONUS INC., Kawasaki-shi, Kanagawa (JP)

(72) Inventor: Hiroyuki SEKIGUCHI, Kawasaki-shi (JP)

(73) Assignee: LUXONUS INC., Kawasaki-shi, Kanagawa (JP)

(21) Appl. No.: 18/688,292

(22) PCT Filed: Aug. 29, 2022
(86) PCT No.: PCT/JP2022/032454

§ 371 Date: Feb. 29, 2024

(30) Foreign Application Priority Data
Sep. 2, 2021 (JP) 2021-143341
Oct. 20, 2021 (JP) 2021-171384

(51) Int. Cl. G06T 19/20; G06T 15/08

(52) U.S. Cl. CPC G06T 19/20; G06T 15/08; G06T 2210/41; G06T 2219/2012; G06T 2219/2016

(57) ABSTRACT

An image processing apparatus including: an internal structure image output unit that constructs an internal structure image, in which color parameters are allowed to vary continuously from a first depth to a second depth, in a depth range of the first depth or more and the second depth or less from a predetermined reference surface, based on volume data indicating a three-dimensional internal structure of an object, and displays the internal structure image on a display device; a profile image output unit that displays on the display device a profile image indicating correspondence relation between a depth from the reference surface and the color parameter; and an input accepting unit that accepts operations of a pointing device, wherein the input accepting unit is configured to accept at least one of the first depth and the second depth, according to the operation content of the pointing device, when the operation position of the pointing device is on the profile image, the internal structure image output unit is configured to reconstruct the internal structure image in which the color parameters in the depth range after change accepted by the input accepting unit are allowed to vary from the first depth to the second depth after change, similarly to the variation aspect of the color parameters before change, and to redisplay the internal structure image on the display device, and the profile image output unit is configured to reconstruct the profile image, according to the first depth and the second depth after change accepted by the input accepting unit, and to redisplay the profile image on the display device.

### TECHNICAL FIELD

The present disclosure relates to an image processing apparatus, an image processing method, and a recording medium.

### BACKGROUND ART

An image generating apparatus that reconstructs images based on signal data of acoustic waves obtained by measuring a predetermined test part of a subject has been developed.

For example, image generating apparatuses that irradiate a subject such as a living body with light from a light source (e.g., laser) to visualize information on the inside of the subject have been actively studied in the medical field. Photoacoustic Tomography (PAT; sometimes also referred to as optical ultrasound tomography, etc.) is one of such optical visualization techniques. In an imaging apparatus utilizing the photoacoustic tomography, irradiated light propagates within the subject, and acoustic waves (typically ultrasonic waves) generated from a light-absorptive biological tissue which has absorbed the energy of the diffused light are detected at a plurality of sites around the subject. Then, the resulting signals are mathematically analyzed and processed to visualize the information related to the optical characteristic values, particularly absorption coefficient distribution, inside the subject. Recently, non-clinical studies for imaging blood vessels of small animals using such photoacoustic tomographic apparatuses, and clinical studies for applying this principle to the diagnostic imaging of breast cancer and the like, or to the preoperative planning in the field of plastic surgery are actively promoted.

Patent Literature 1 discloses a technique that enables estimation of the interface of a subject using image data. In addition, Patent Literature 2 discloses a technique to improve the accuracy of separating superficial blood vessels and body hairs in an internal structure image reconstructed from the photoacoustic signals.

### CITED LIST

### Patent Literature

Patent Literature 1: WO 2018/110558
Patent Literature 2: Japanese Patent Laid-Open Publication No. 2019-25251

### SUMMARY OF DISCLOSURE [Problem to be Solved by the Disclosure]

An object of the present disclosure is to provide a graphical user interface (GUI) that makes it easier to recognize the internal structure of an object including a living body.

### Solution to Problem

According to an aspect of the present disclosure,
there is provided an image processing apparatus, including:
an internal structure image output unit that constructs an internal structure image, in which color parameters are allowed to vary continuously from a first depth to a second depth, in a depth range of the first depth or more and the second depth or less from a predetermined reference surface, based on volume data indicating a three-dimensional internal structure of an object, and displays the internal structure image on a display device;
a profile image output unit that displays on the display device a profile image indicating correspondence relation between a depth from the reference surface and the color parameter; and
an input accepting unit that accepts operations of a pointing device,
wherein
the input accepting unit is configured to accept a change in at least one of the first depth and the second depth, according to an operation content of the pointing device, when an operation position of the pointing device is on the profile image,
the internal structure image output unit is configured to reconstruct the internal structure image, in which color parameters in the depth range after change accepted by the input accepting unit are allowed to vary from the first depth to the second depth after change, similarly to a variation aspect of the color parameters before change, and to redisplay the internal structure image on the display device, and
the profile image output unit is configured to reconstruct the profile image, according to the first depth and the second depth after change accepted by the input accepting unit, and to redisplay the profile image on the display device.

### Advantageous Effects of the Disclosure

The present disclosure provides a graphical user interface (GUI) that makes it easier to recognize the internal structure of an object including a living body.

### BRIEF DESCRIPTION OF DRAWINGS

FIG. 1 is a schematic configuration diagram of an image processing apparatus 10 according to a first embodiment of the present disclosure.

FIG. 2 is a diagram illustrating an example of an internal structure image 30 and a profile image 40, displayed on a display device 20 according to the first embodiment of the present disclosure.

FIG. 3 is a schematic configuration diagram of an image processing apparatus 10 according to a modified example 1 of the first embodiment of the present disclosure.

FIG. 4 is a diagram illustrating an example of an internal structure image 30, a profile image 40, a first slider image 60, and a second slider image 70, displayed on a display device 20 according to the modified example 1 of the first embodiment of the present disclosure.

FIG. 5A is a diagram illustrating an example of an internal structure image 30 and a profile image 40, before a depth range is changed, according to an operation example of a graphical interface of the present disclosure.

FIG. 5B is a diagram illustrating an example of the internal structure image 30 and the profile image 40, after the depth range is changed, according to the operation example of the graphical interface of the present disclosure.

FIG. 5C is a diagram illustrating another example of the internal structure image 30 and the profile image 40, after the depth range is changed, according to the operation example of the graphical interface of the present disclosure.

FIG. 5D is a diagram illustrating another example of the internal structure image 30 and the profile image 40, after the depth range is changed, according to the operation example of the graphical interface of the present disclosure.

FIG. 6 is a flowchart giving an outline of an image processing method with the image processing apparatus 10 according to the first embodiment of the present disclosure.

FIG. 7 is a schematic configuration diagram of an image processing apparatus 10 according to a modified example 2 of the first embodiment of the present disclosure.

### DESCRIPTION OF EMBODIMENTS

### First Embodiment of the Present Disclosure

A first embodiment of the present disclosure will be described below with reference to the drawings. The present disclosure is not limited to these exemplifications, but intended to be indicated by claims and encompass all the changes which fall within the meaning and scope equivalent to claims.

### (1) Configuration of Image Processing Apparatus 10

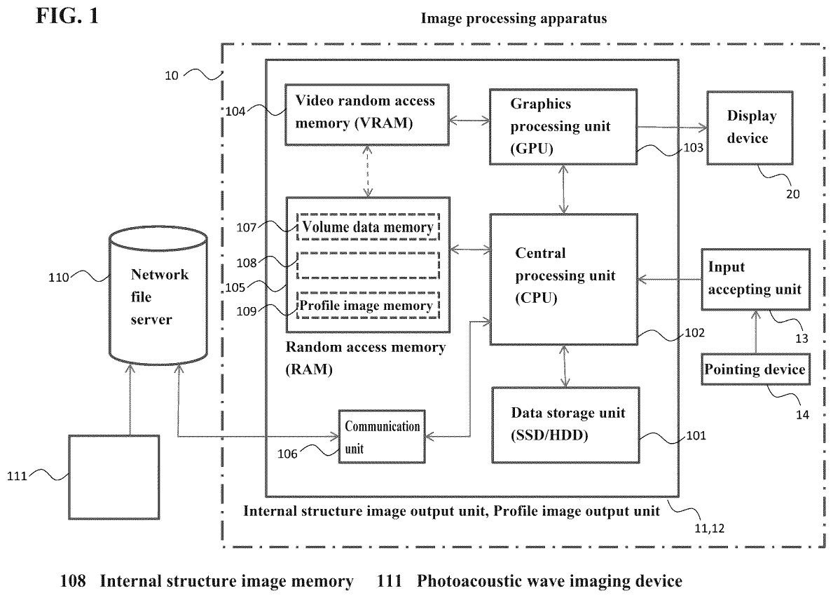

An image processing apparatus 10 according to this embodiment is configured to process volume data indicating a three-dimensional internal structure of a biological tissue, for example, to make it easier for a user to recognize the internal structure of the biological tissue of a subject. FIG. 1 is a schematic configuration diagram of the image processing apparatus 10 according to this embodiment. As illustrated in FIG. 1, an image processing apparatus 10 includes, for example, an internal structure image output unit 11, a profile image output unit 12, and an input accepting unit 13. As illustrated in FIG. 1, the internal structure image output unit 11 and the profile image output unit 12 may be part of a computer having, for example, a data storage unit (SSD/HDD) 101, a central processing unit (CPU) 102, a graphics processing unit (GPU) 103, a video random access memory (VRAM) 104, a random access memory (RAM) 105, and a communication unit 106. In this embodiment, the volume data is obtained by image reconstruction with signal data of the photoacoustic waves generated by light irradiation to a living body. A user means a person who operates the image processing apparatus 10.

The SSD/HDD 101 is configured, for example, to store data such as a program related to a photoacoustic wave measurement. The CPU 102 is configured, for example, to control each unit of the image processing apparatus 10 by executing a predetermined program stored in the SSD/HDD 101. The GPU 103 is configured, for example, to execute a program related to an image processing in corporation with the CPU 102. The VRAM 104 is configured, for example, to temporarily hold the information, program or the like processed by the GPU 103. The RAM 105 is configured, for example, to have a volume data memory (region) 107, an internal structure image memory (region) 108, a profile image memory (region) 109, and the like, and to temporarily hold the information, program, or the like processed by the CPU 102. The communication unit 106 is configured, for example, to obtain information required for image processing from a network file server 110. The network file server 110 is configured, for example, to store imaging data taken with a photoacoustic wave imaging device 111.

The internal structure image output unit 11 is configured to construct an internal structure image 30, in which color parameters are allowed to vary continuously from a first depth to a second depth, in a depth range of the first depth or more and the second depth or less from a predetermined reference surface, based on the volume data indicating a three-dimensional internal structure of an object (a biological tissue in this embodiment), and to display the internal structure image on the display device 20. Further, the internal structure image output unit 11 is configured to construct the internal structure image 30 by using the same color parameters of the internal structure image 30 constructed based on the volume data within a range of less than the first depth as the color parameters at the first depth and using the same color parameters of the internal structure image 30 constructed based on the volume data within a range of more than the second depth as the color parameters at the second depth, and to display the internal structure image on the display device 20.

The predetermined reference surface means a flat surface of a predetermined reference height, or a continuous surface defining the interface of a subject. The internal structure image output unit 11 is preferably configured such that the predetermined reference surface can be switched to the flat surface of the predetermined reference height or to the continuous surface defining the interface of the subject to reconstruct the internal structure image 30 and to redisplay the internal structure image on the display device 20. In order to determine the continuous surface defining the interface of the subject, a known method such as cross simulation described in Patent Literature 1 can be used, for example.

The first depth means, for example, the lower depth limit (the shallowest position) of the depth range, and the second depth means, for example, the upper depth limit (the deepest position) of the depth range.

The volume data is three-dimensional data as a collection of voxels, where the voxel means a unit region having information on physical properties such as density, mass, and oxygen saturation of a unit space region of the subject as predetermined physical parameters. In other words, each voxel includes coordinate values and physical parameters, and each voxel in the volume data and the unit space region of the subject correspond to each other. The internal structure image 30 also means a two-dimensional image constructed by a collection of pixels for display.

The color parameter is a parameter that includes at least one of attributes: hue, chroma, and brightness. The color parameters of each pixel in the internal structure image 30 vary continuously, for example, from red (shallow) to blue (deep), depending on the depth of each voxel corresponding to each pixel from the reference surface. The internal structure image output unit 11 is preferably configured so that it can switch which attribute among the attributes included in the color parameter (hue, chroma, and brightness) is to be allowed to vary continuously according to the depth from the reference surface.

The internal structure image 30 refers to an image for two-dimensional display, constructed based on the volume data indicating the internal structure of the biological tissue. In this embodiment, a case will be described where the internal structure image 30 is a photoacoustic image constructed based on signal data of photoacoustic waves.

The display device 20 is configured, for example, to be connected to the image processing apparatus 10 and to display the internal structure image 30 and the like by executing a predetermined program. The display device 20 may be a two-dimensional display device or a stereoscopic display device. Specific examples of the display device 20 include liquid crystal displays, organic EL (OLED) displays, head-mounted displays, direct-view type stereoscopic displays. The display device 20 may be part of the image processing apparatus 10.

The profile image output unit 12 is configured to display on the display device 20 a profile image 40 indicating correspondence relation between the depth from the reference surface and the color parameters, together with the internal structure image 30.

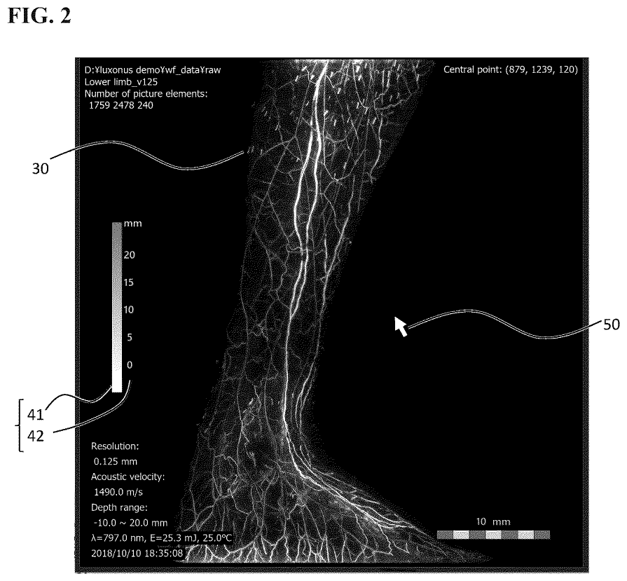

FIG. 2 is a diagram illustrating an example of an internal structure image 30 and a profile image 40, displayed on a display device 20 according to this embodiment. As illustrated in FIG. 2, in this embodiment, the profile image 40 includes a color bar 41 and an index 42. The color bar 41 is, for example, a vertically long bar-shaped image, and the color parameters vary from the upper end to the lower end, similarly to the internal structure image 30. In the color bar 41, for example, the lower end corresponds to the first depth, and the upper end corresponds to the second depth. The index 42 is, for example, a group of multiple numerical values adjacent to the color bar 41, indicating the correspondence relation between the color parameter of the color bar 41 and the depth from the reference surface. For example, in the group of multiple numerical values of the index 42, the lowermost numerical value indicates the first depth, and the uppermost numerical value indicates the second depth. A user can recognize how deep the region which the user has an interest in (hereinafter referred to as a region of interest) is located from the reference surface in the internal structure image 30 by checking the internal structure image 30 and the profile image 40 displayed on the display device 20. Note that for the image processing apparatus 10 of this embodiment, there are two ways to set the reference surface which is at a depth of zero. One of them is to set a position on the skin surface of the subject to zero. The position on the skin surface can be determined, for example, by a cross simulation processing described in Patent Literature 1. The other one is to arrange multiple sensors in a bowl shape surrounding the subject, and to set the curvature center position of the sensor to z=0, with respect to the central axis (z axis) of the bowl. When using this reference, the height of the shallowest detection surface is about −10 mm, and the photoacoustic wave sensor of this embodiment has the highest resolution in a range from −10 mm to +10 mm.

The input accepting unit 13 is configured to accept operations of the pointing device 14. In this specification, the pointing device 14 refers to, for example, an input device for operating a pointer 50 displayed on the display device 20, and specifically includes a mouse, a trackball, a touch panel (a touch sensor that detects position designation, drag, etc., in response to touch input by a user).

### (2) Graphical Interface Included in Image Processing Apparatus 10

The image processing apparatus 10 according to this embodiment includes a graphical user interface (GUI) that makes it easier to recognize the internal structure of the biological tissue. For example, the user operates the pointer 50 displayed on the display device 20 using the pointing device 14. The image processing apparatus 10 is configured to perform various processes in response to the operation position and operation content of the pointing device 14. The following is an explanation of some of functions of the GUI included in the image processing apparatus 10 according to this embodiment. In this specification, the operation content of the pointing device 14 means, for example, various operations such as clicking, dragging, and wheel running, and the operation position of the pointing device 14 means the position of the pointer 50 at the beginning of the above-described operations.

### (2-1) Function to Move Image

The GUI included in the image processing apparatus 10 according to this embodiment can move each of the internal structure image 30 and the profile image 40 displayed on the display device 20 to an arbitrary position on the display device 20.

The internal structure image output unit 11 is configured to display the internal structure image 30 in a movable aspect, according to the operation content of the pointing device 14 accepted by the input accepting unit 13, when the operation position of the pointing device 14 accepted by the input accepting unit 13 is on the internal structure image 30. Specifically, the internal structure image output unit 11 may be configured, for example, to move the internal structure image 30 in the direction in which the pointer 50 moves when a drag operation is performed on the internal structure image 30.

The profile image output unit 12 is configured to display the profile image 40 in a movable aspect, according to the operation content of the pointing device 14 accepted by the input accepting unit 13, when the operation position of the pointing device 14 accepted by the input accepting unit 13 is on the profile image 40. Specifically, the profile image output unit 12 may be configured, for example, to move the profile image 40 in the direction in which the pointer 50 moves when a drag operation is performed on the profile image 40. The user can move the profile image 40 to an arbitrary position as needed, which makes it easier to recognize how deep the region of interest is located from the reference surface, for example. In this embodiment, the operation position of the pointing device 14 being on the profile image 40 means, for example, the operation of the pointing device 14 is started while the pointer 50 is located on the color bar 41 (or on the index 42).

### (2-2) Function to Enlarge or Reduce Image

The GUI included in the image processing apparatus 10 according to this embodiment can enlarge or reduce the internal structure image 30 displayed on the display device 20.

The internal structure image output unit 11 is configured to display the internal structure image 30 in an aspect that enables at least one of enlargement and reduction, according to the operation content of the pointing device 14 accepted by the input accepting unit 13, when the operation position of the pointing device 14 accepted by the input accepting unit 13 is on the internal structure image 30. Specifically, the internal structure image output unit 11 may be configured, for example, to enlarge the internal structure image 30 when the wheel is operated in one direction (e.g., upward) on the internal structure image 30, and to reduce the internal structure image 30 when the wheel is operated in the other direction (e.g., downward).

### (2-3) Function to Change Depth Range

The GUI included in the image processing apparatus 10 according to this embodiment can change at least one of the first depth and the second depth, and redisplay each of the internal structure image 30 and the profile image 40 displayed on the display device 20.

The input accepting unit 13 is configured to accept at least one of the first depth and the second depth, according to the operation content of the pointing device 14, when the operation position of the pointing device 14 is on the profile image 40.

The internal structure image output unit 11 is configured to reconstruct the internal structure image 30 in which the color parameters of each pixel in the depth range after change accepted by the input accepting unit 13 are allowed to vary, from the first depth to the second depth after change, similarly to the variation aspect of the color parameters before change, and to redisplay the internal structure image on the display device 20. In this specification, “variation aspect” of the color parameter indicates which attribute among attributes included in the color parameter varies continuously. The color parameter being changed similarly to the variation aspect of the color parameter before change means, for example, when the color parameter before change varies from red to blue over a range from the first depth to the second depth, the color parameter after change is allowed to vary similarly in that it varies from red to blue.

The profile image output unit 12 is configured to reconstruct the profile image 40, according to the first depth and the second depth after change accepted by the input accepting unit 13, and to redisplay the profile image on the display device 20. As described above, the variation aspect of the color parameter is not changed before and after changing the depth range. Accordingly, in reconstructing the profile image 40, only the index 42 has to be changed, leaving the image of the color bar 41 unchanged. The user can adjust the information on the region of interest in the depth direction so as to make it easier to visually recognize it by simple operation, operating the pointing device 14.

### (2-4) Function to Change First Depth (or Second Depth)

The function to change the depth range explained in (2-3) can be classified in more detail. For example, the GUI included in the image processing apparatus 10 according to this embodiment can change only the first depth (or the second depth) and redisplay each of the internal structure image 30 and the profile image 40 displayed on the display device 20.

The input accepting unit 13 is configured to accept the change in at least one of the first depth and the second depth, according to the operation content of the pointing device 14, when the operation position of the pointing device 14 is on the first region of the profile image 40. The first region of the profile image 40 means, for example, a lower part (e.g., one-fourth the length from the lower end) of the color bar 41. Specifically, the input accepting unit 13 may be configured, for example, to accept a change which increases only the first depth when the wheel is operated in one direction (e.g., upward) and decreases only the first depth when the wheel is operated in the other direction (e.g., downward), on the lower part of the color bar 41.

The input accepting unit 13 is configured to accept a change in the other one, other than the one described above, of the first depth and the second depth, according to the operation content of the pointing device 14, when the operation position of the pointing device 14 is on the second region of the profile image 40. The second region of the profile image 40 means, for example, an upper part (e.g., one-fourth the length from the upper end) of the color bar 41. Specifically, the input accepting unit 13 may be configured, for example, to accept a change which increases only the second depth when the wheel is operated in one direction (e.g., upward) and decreases only the second depth when the wheel is operated in the other direction (e.g., downward), on the upper part of the color bar 41.

### (2-5) Function to Shift Depth Range

The GUI included in the image processing apparatus 10 according to this embodiment can change the first depth and the second depth to shift the depth range, and redisplay each of the internal structure image 30 and the profile image 40 displayed on the display device 20.

The input accepting unit 13 is configured to accept the change to move the depth range in at least one of the deep direction and the shallow direction, according to the operation content of the pointing device 14, without changing the size of the depth range, when the operation position of the pointing device 14 is on the third region of the profile image 40. The third region of the profile image 40 means, for example, a central part (e.g., one-fourth the length extending upward/downward from the center) of the color bar 41. Specifically, the input accepting unit 13 may be configured, for example, to accept a change which increases the first depth and the second depth by the same value when the wheel is operated in one direction (e.g., upward) and decreases the first depth and the second depth by the same value when the wheel is operated in the other direction (e.g., downward), on the central part of the color bar 41.

### (2-6) Function to Enlarge or Reduce Depth Range

The GUI included in the image processing apparatus 10 according to this embodiment can change the first depth and the second depth so that the depth range is enlarged or reduced, and redisplay each of the internal structure image 30 and the profile image 40 displayed on the display device 20. When there is no variation in each of the color parameters at the first depth and the second depth, enlarging the depth range will reduce the variation in the color parameter per unit depth, which enables test over a wide depth range. Similarly, when there is no variation in the color parameter of each of the first depth and the second depth, reducing the depth range will increase the variation in the color parameter per unit depth, which allows depth differences to be read in detail within a narrow depth range.

The input accepting unit 13 is configured to accept the change that enables at least one of enlargement and reduction of the depth range, according to the operation content of the pointing device 14, without changing the center of the depth range, when the operation position of the pointing device 14 is on the profile image 40. Specifically, the input accepting unit 13 may be configured, for example, to accept a change that increases the first depth and decreases the second depth by the same value as the increase in the first depth to reduce the depth range, when the wheel is operated in one direction (e.g., upward) while right-clicking, and a change that decreases the first depth and increases the second depth by the same value as the decrease in the first depth to enlarge the depth range, when the wheel is operated in the other direction (e.g., downward), on the profile image 40.

### (3) Effect Obtained by this Embodiment

According to this embodiment, one or more than one effects described below are obtained.

(a) The GUI included in the image processing apparatus 10 according to this embodiment can move each of the internal structure image 30 and the profile image 40 displayed on the display device 20 to an arbitrary position on the display device 20. This function enables the user to move the profile image 40 to an arbitrary position as needed, which makes it easier to recognize how deep the region of interest is located from the reference surface, for example.

(b) The GUI included in the image processing apparatus 10 according to this embodiment can enlarge or reduce the internal structure image 30 displayed on the display device 20. This function enables the user to enlarge or reduce the internal structure image 30 as needed, which makes it easier to recognize how deep the region of interest is located from the reference surface, for example.

(c) The GUI included in the image processing apparatus 10 according to this embodiment can change at least one of the first depth and the second depth, and redisplay each of the internal structure image 30 and the profile image 40 displayed on the display device 20. This function enables the user to adjust the information on the region of interest in the depth direction to visually recognize it with more ease by simple operation, operating the pointing device 14.

(d) The GUI included in the image processing apparatus 10 according to this embodiment can change only the first depth (or the second depth) and redisplay each of the internal structure image 30 and the profile image 40 displayed on the display device 20. This function enables the user to adjust the information on the region of interest in the depth direction to visually recognize it with more ease by simple operation, operating the pointing device 14.

(e) The GUI included in the image processing apparatus 10 according to this embodiment can change the first depth and the second depth so that the depth range is shifted, and redisplay each of the internal structure image 30 and the profile image 40 displayed on the display device 20. This function enables the user to adjust the information on the region of interest in the depth direction to visually recognize it with more ease by simple operation, operating the pointing device 14.

(f) The GUI included in the image processing apparatus 10 according to this embodiment can change the first depth and the second depth so that the depth range is enlarged or reduced, and redisplay each of the internal structure image 30 and the profile image 40 displayed on the display device 20. This function enables the user to adjust the information on the region of interest in the depth direction to visually recognize it with more ease by simple operation, operating the pointing device 14.

(g) In this embodiment, the volume data indicating the internal structure of the biological tissue is obtained by image reconstruction with signal data of the photoacoustic waves generated by light irradiation to a living body. With the photoacoustic tomography that can visualize, for example, only blood vessels and can provide an internal structure image 30 displaying foreground blood vessels and background blood vessels simultaneously, the present disclosure can be particularly effectively applied.

### (4-1) Modified Example 1 of First Embodiment

The embodiment described above can be modified as needed, similarly to the following modified examples. Hereinafter, only elements that differ from those in the embodiment descried above will be explained, and elements that are substantially the same as those in the embodiment described above will be marked with the same reference numerals and their explanation will be omitted.

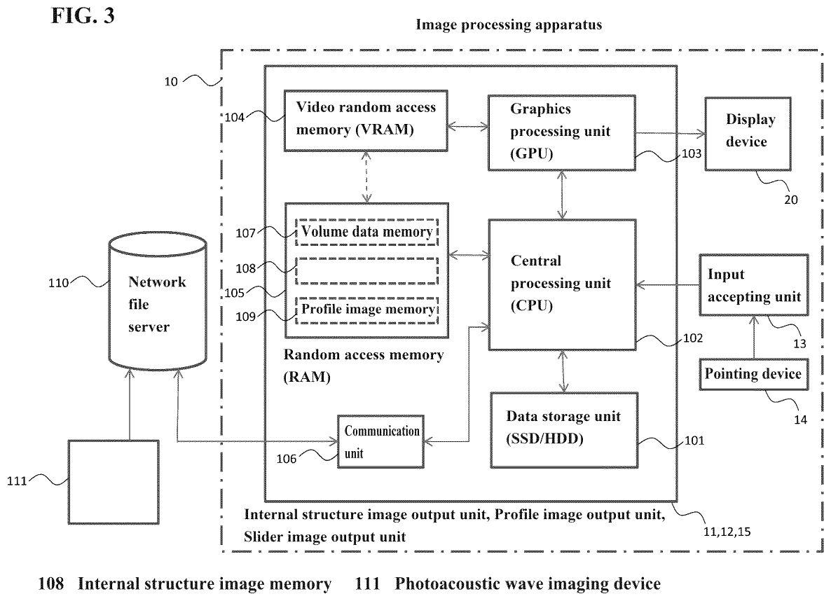

FIG. 3 is a schematic configuration diagram of the image processing apparatus 10 according to this modified example. As illustrated in FIG. 3, the image processing apparatus 10 according to this modified example may further include, for example, a slider image output unit 15. The slider image output unit 15 may be part of a computer having CPU 102 or the like, similarly to the internal structure image output unit 11 and the profile image output unit 12. The slider image output unit 15 is configured, for example, to display on the display device 20 the first slider image 60 indicating the position and size of the depth range (the first depth or more and the second depth or less) in which the color parameters vary continuously, with respect to the whole depth range (also referred to as imaging range) of the three-dimensional volume data obtained by imaging, and the second slider image 70 indicating the depth position of the display range (in the depth direction) and the size of the depth range of the internal structure image 30 displayed on the display device 20, with respect to the whole depth range of the volume data, together with the internal structure image 30 and the profile image 40.

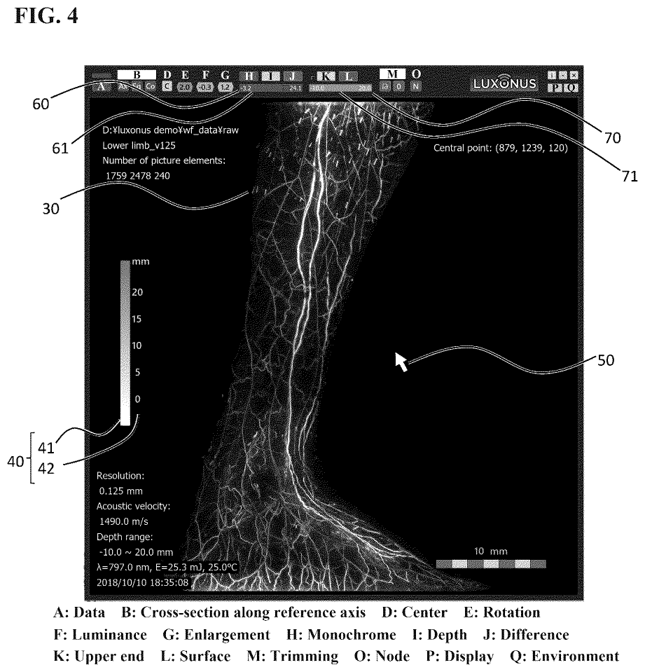

FIG. 4 is a diagram illustrating an example of an internal structure image 30, a profile image 40, a first slider image 60, and a second slider image 70, displayed on a display device 20 according to this modified example. As illustrated in FIG. 4, the first slider image 60 is, for example, a horizontally long bar-shaped image, and has a first slider bar 61 in the image. Further, the second slider image 70 is, for example, a horizontally long bar-shaped image, and has a second slider bar 71 in the image. In the first slider image 60 and the second slider image 70, one end (e.g., left end) represents the lower depth limit (the shallowest position) of the depth range of the volume data, while the other end (e.g., right end) represents the upper depth limit (the deepest position) of the depth range of the volume data. In the first slider bar 61, the one end (e.g., left end) corresponds to the first depth, while the other end (e.g., right end) corresponds to the second depth. In the second slider bar 71, the one end (e.g., left end) corresponds to the lower depth limit of the display range of the internal structure image 30, while the other end (e.g., right end) corresponds to the upper depth limit of the display range of the internal structure image 30.

The slider image output unit 15 is configured to reconstruct the first slider image 60, according to the first depth and the second depth after change accepted by the input accepting unit 13, and redisplay the first slider image on the display device 20. Specifically, the slider image output unit 15 may be configured to change the position and size of the first slider bar 61, according to the first depth and the second depth after change. The user may check the first slider image 60 as needed, so as to more easily recognize where the depth range in which the color parameters are allowed to vary continuously is located in the whole depth range of the volume data.

Similar to the profile image 40, the first slider image 60 can be used as part of the GUI having a function to change the depth range. In this event, the input accepting unit 13 may be configured to accept a change in at least one of the first depth and the second depth, according to the operation content of the pointing device 14, when the operation position of the pointing device 14 is on the first slider image 60. However, from the viewpoint of ability of changing the depth range while visually confirming the correspondence relation between the depth from the reference surface and the color parameter, it is preferable to operate the pointing device 14 on the profile image 40, similarly to the above-described first embodiment.

The second slider image 70 can be used as part of the GUI having function to change the display depth range of the internal structure image 30. In this event, the input accepting unit 13 may be configured to accept the change in at least one of the lower depth limit and the upper depth limit of the display depth range of the internal structure image 30, according to the operation content of the pointing device 14, when the operation position of the pointing device 14 is on the second slider image 70. The internal structure image output unit 11 may also be configured to reconstruct the internal structure image 30, according to the display depth range of the internal structure image 30 after change accepted by the input accepting unit 13, and to redisplay the internal structure image on the display device 20. Further, the slider image output unit 15 may be configured to reconstruct the second slider image 70, according to the display depth range of the internal structure image 30 after change accepted by the input accepting unit 13, and to redisplay the internal structure image on the display device 20. Specifically, the slider image output unit 15 may be configured to change the position and size of the second slider bar 71, according to the display depth range of the internal structure image 30 after change. The user may change the display depth range of the internal structure image 30 as needed, for example, to hide voxels in unnecessary regions displayed in the foreground, which makes it easier to recognize the information on the region of interest.

### (4-2) Modified Example 2 of First Embodiment

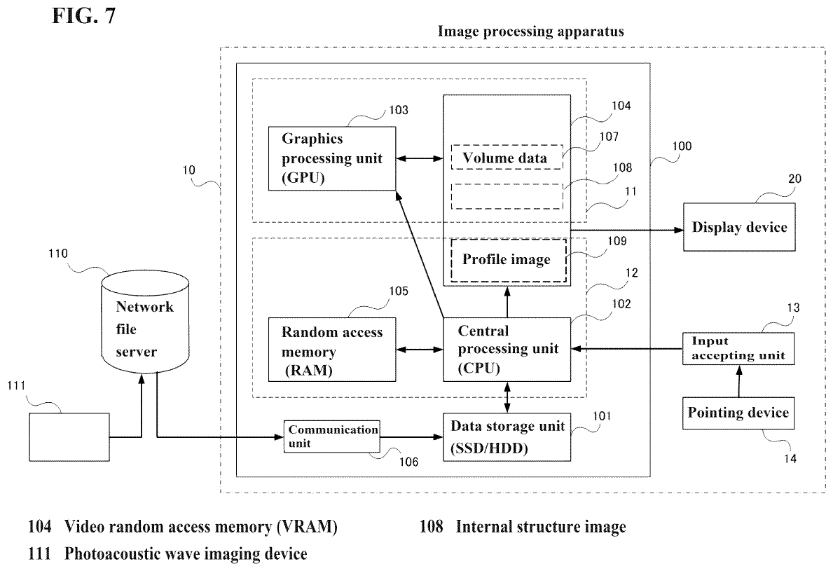

FIG. 7 is a schematic configuration diagram of the image processing apparatus 10 according to this modified example. As illustrated in FIG. 7, the image processing apparatus 10 of this modified example includes, for example, an image processor 100, a display device 20, an input accepting unit 13, and a pointing device 14. The image processor 100 includes, for example, an internal structure image output unit 11, a profile image output unit 12, a data storage unit (SSD/HDD) 101, and a communication unit 106. The internal structure image output unit 11 includes, for example, a graphics processing unit (GPU) 103, a video random access memory (VRAM) 104, a volume data region 107 in the VRAM 104, and an internal structure image region 108. The profile image output unit 12 includes, for example, a central processing unit (CPU) 102, a random access memory (RAM) 105, a VRAM 104, and a profile image region 109 in the VRAM 104. The resulting volume data taken with the photoacoustic wave imaging device 111 is accumulated in the network file server 110. When the user wants to observe the volume data, the volume data is stored in the volume data region 107 in the VRAM 104 via the communication unit 106, the data storage unit 101, and the CPU 102. The processing of converting the volume data into the internal structure image and the processing of the internal structure image according to a user's instruction via the input accepting unit 13, which has been sent to the GPU 103 via the CPU 102, are performed and stored in the internal structure image region 108. The information on the profile image 40 is written directly in the profile image region 109 in the VRAM 104 by the CPU 102. Contents of the internal structure image region 108 and the profile image region 109 are displayed on the display device 20.

The image processor 100 includes the components described below. The data storage unit 101 is configured, for example, to store data such as a program related to the photoacoustic wave measurement. The CPU 102 is configured, for example, to control each unit of the image processing apparatus 10 by executing a predetermined program stored in the data storage unit 101. The GPU 103 is configured, for example, to execute a program related to an image processing, in corporation with the CPU 102. The VRAM 104 is a memory for displaying on the display device 20 the image information processed by the GPU 103 and the CPU 102, for example. The VRAM 104 is also used as a working memory for image processing in the GPU 103. The RAM 105 is a working memory for the CPU 102, and is configured, for example, to temporarily hold the information, program, and the like, processed by the CPU 102. The communication unit 106 is configured, for example, to obtain information required for image processing from the network file server 110 and to accumulate it in the data storage unit 101. The network file server 110 is configured, for example, to store imaging data taken with a photoacoustic wave imaging device 111.

### (5) Operation Examples of Graphical Interface

Next, operation examples of the graphical interface according to the present disclosure will be described. The following operation examples are illustrative of the present disclosure, and the present disclosure is not limited by these operation examples.

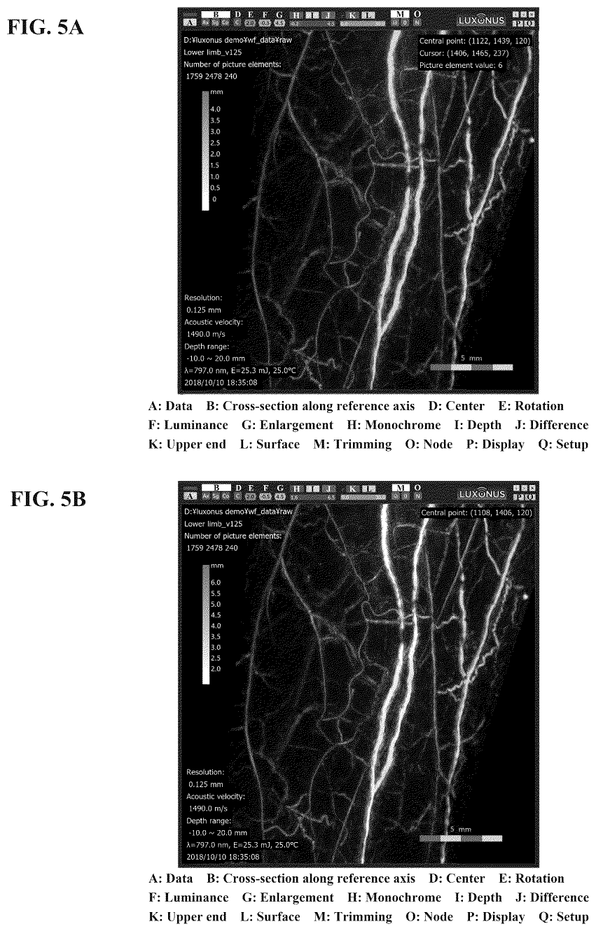

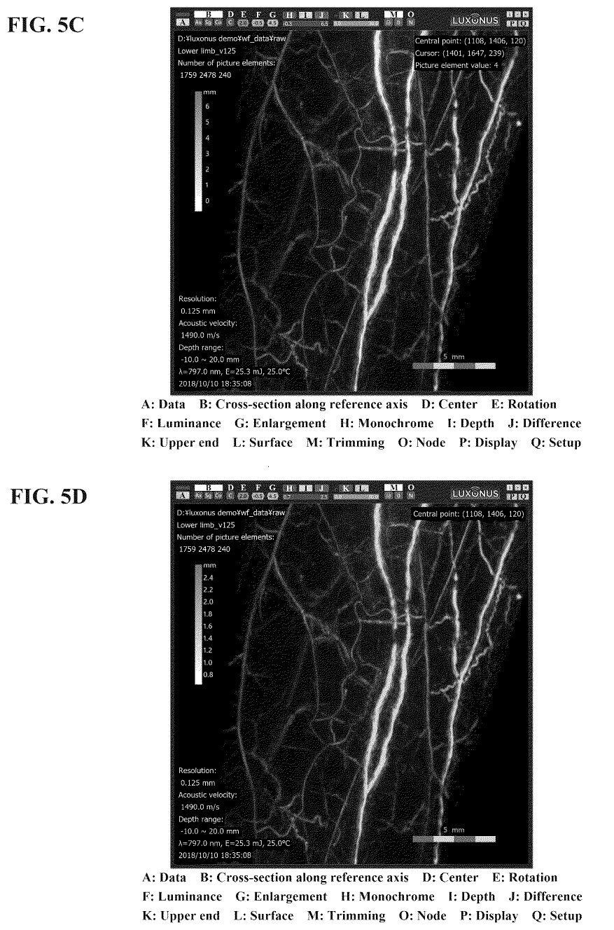

FIG. 5A is a diagram illustrating an example of an internal structure image 30 and a profile image 40 before a depth range is changed, according to this operation example. In this operation example, the internal structure image 30 is a photoacoustic image illustrating internal blood vessels of the subject's leg. The reference surface is an example of a continuous surface specified by the cross simulation described above, with respect to the vicinity of the skin surface of the subject as a reference position.

As illustrated in FIG. 5A, the first depth is 0 mm and the second depth is 4 mm, before the depth range is changed. The user checks the color parameters of the internal structure image 30 and the profile image 40 in correspondence with each other to recognize, for example, how deep a blood vessel, present in a region of interest in a depth range of the first depth or more and the second depth or less, is located from the reference surface.

For example, when the region of interest is a deeper range (e.g., about 5 mm), the user may operate the pointing device 14 to move the pointer 50 to the center of the color bar 41, and then operate the wheel upward. The above-described operation can increase the first depth and the second depth by the same value, and change (shift) the depth range. FIG. 5B illustrates the internal structure image 30 and the profile image 40 after the depth range is changed by the above-described operation. As illustrated in FIG. 5B, the first depth is 2 mm and the second depth is 6 mm, after the depth range is changed. The user checks the color parameters of the internal structure image 30 and the profile image 40, which are redisplayed, in correspondence with each other to recognize, for example, how deep a blood vessel, present in a region of interest in a deeper range, is located from the reference surface.

In an example illustrated in FIG. 5B, however, the depth range has been shifted, so that, for example, for a blood vessel present in a depth range of 0 mm or more and 2 mm or less from the reference surface, variations in color parameters are lost in the internal structure image 30 indicating the depth of each voxel constituting the blood vessel region, resulting in constant color parameters (having the same value as that of the first depth after change). Thus, it becomes difficult for the user to recognize how deep the blood vessel present in this range is located from the reference surface. For example, when a user wants to maintain the variation in color parameters in a shallow range while the region of interest is in a deeper range, the user may operate the pointing device 14 to move the pointer 50 to the upper part of the color bar 41, and operate the wheel upward. The above-described operation can increase only the second depth, and change the depth range. FIG. 5C illustrates the internal structure image 30 and the profile image 40 after the depth range is changed by the above-described operation. As illustrated in FIG. 5C, the first depth is 0 mm and the second depth is 6 mm, after the depth range is changed. The user checks the color parameters of the internal structure image 30 and the profile image 40, which are redisplayed, in correspondence with each other to recognize, for example, how deep a blood vessel, present in a region of interest in a deeper range, and a blood vessel, present in a depth range before change, are located from the reference surface.

For example, when the region of interest is a narrower depth range, the user may operate the pointing device 14 to move the pointer 50 onto the color bar, and operate the wheel upward while right-clicking. The above-described operation can increase the first depth, and decrease the second depth by the same value as the increase in the first depth to change (reduce) the depth range. FIG. 5D illustrates the internal structure image 30 and the profile image 40 after the depth range is changed by the above-described operation. As illustrated in FIG. 5D, the first depth is 0.8 mm and the second depth is 2.4 mm, after the depth range is changed. The user checks the color parameters of the internal structure image 30 and the profile image 40, which are redisplayed, in accordance with each other to recognize, for example, how deep a blood vessel, present in a region of interest in a narrower depth range, is located from the reference surface.

Thus, it is confirmed that the user can adjust the information on the biological tissue in the depth direction to be visually recognized with more ease using the GUI included in the image processing apparatus 10.

### (6) Image Processing Method with Image Processing Apparatus 10

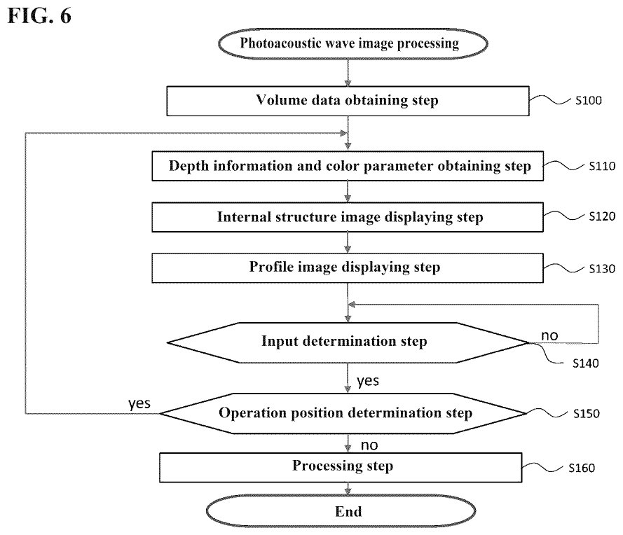

Next, the image processing method with the image processing apparatus 10 will be described. FIG. 6 is a flowchart giving an outline of an image processing method with the image processing apparatus 10 according to the first embodiment of the present disclosure. As illustrated in FIG. 6, the image processing method of this embodiment includes, for example, a volume data obtaining step S100, a depth information and color parameter obtaining step S110, an internal structure image displaying step S120, a profile image displaying step S130, an input determination step S140, an operation position determination step S150, and a processing step S160.

In the volume data obtaining step S100, for example, the photoacoustic wave imaging device 111 is activated, and photoacoustic wave imaging is performed, to obtain volume data indicating a three-dimensional internal structure of an object (e.g., biological tissue).

In the depth information and color parameter obtaining step S110, for example, information on the first depth and the second depth from a predetermined reference surface and color parameters of an internal structure image 30 constructed based on volume data in a depth range from the first depth to the second depth are obtained.

In the internal structure image displaying step S120, for example, the internal structure image 30, in which the color parameters are allowed to vary continuously from the first depth to the second depth, is constructed based on the volume data and the color parameters, and displayed on the display device 20.

In the profile image displaying step S130, for example, the profile image 40 indicating the correspondence relation between the depth from the reference surface and the color parameter is displayed on the display device 20.

In the input determination step S140, for example, presence or absence of an input from the pointing device 14 is determined. When an input from the pointing device 14 is present, an operation position determination step S150 is performed. When an input from the pointing device 14 is absent, the input determination step S140 may be performed again.

In the operation position determination step S150, for example, it is determined whether the operation position of the pointing device 14 is on the profile image 40 or not. When the operation position of the pointing device 14 is on the profile image 40, for example, the depth information and color parameter obtaining step S110 may be performed again. When the operation position of the pointing device 14 is not on the profile image 40, the processing step S160 may be performed.

In the processing step S160, other processing appropriate to the operation content of the pointing device 14 is performed.

The above-described steps can perform image processing that makes it easier to recognize the internal structure of a biological tissue. The present disclosure is also applicable as a program that makes a computer execute the above-described steps (procedures) and as a computer readable recording medium that records the program.

### Other Embodiments of the Present Disclosure

The embodiment of the present disclosure is specifically described above. However, the present disclosure is not limited to the above-described embodiment and can be variously changed without departing from the gist of the present disclosure.

For example, in the above-described embodiment, an explanation has been given for a case where the volume data indicating the internal structure of the biological tissue includes signal data of the photoacoustic waves generated by light irradiation to a living body and the internal structure image 30 is a photoacoustic image constructed based on the signal data of photoacoustic waves. However, the internal structure image 30 is not limited to the photoacoustic image. The internal structure image 30 may be, for example, an ultrasonic image constructed based on the data obtained by the ultrasonic diagnostic imaging method, or an MRI angiographic image constructed based on the data obtained by the MRI angiographic diagnostic method. In the above-described embodiments, an explanation has also been given for a case where volume data indicating the three-dimensional internal structure of the biological tissue is processed. However, the present disclosure is also applicable to analyzers and the like that use ultrasound to analyze the internal structures of not only living bodies but also roads.

### Preferable Aspects of the Present Disclosure

Preferable aspects of the present disclosure are supplementarily described below.

### (Supplementary Description 1)

An image processing apparatus, including:
an internal structure image output unit that constructs an internal structure image, in which color parameters are allowed to vary continuously from a first depth to a second depth, in a depth range of the first depth or more and the second depth or less from a predetermined reference surface, based on volume data indicating a three-dimensional internal structure of an object, and displays the internal structure image on a display device;
a profile image output unit that displays on the display device a profile image indicating correspondence relation between a depth from the reference surface and the color parameter; and
an input accepting unit that accepts operations of a pointing device,
wherein
the input accepting unit is configured to accept a change in at least one of the first depth and the second depth, according to an operation content of the pointing device, when an operation position of the pointing device is on the profile image,
the internal structure image output unit is configured to reconstruct the internal structure image, in which color parameters in the depth range after change accepted by the input accepting unit are allowed to vary, from the first depth to the second depth after change, similarly to a variation aspect of the color parameters before change, and to redisplay the internal structure image on the display device, and
the profile image output unit is configured to reconstruct the profile image, according to the first depth and the second depth after change accepted by the input accepting unit, and to redisplay the profile image on the display device.

### (Supplementary Description 2)

The image processing apparatus according to Supplementary Description 1,
wherein the internal structure image output unit is configured to display the internal structure image in a movable aspect, according to the operation content of the pointing device accepted by the input accepting unit, when the operation position of the pointing device accepted by the input accepting unit is on the internal structure image, and
the profile image output unit is configured to display the profile image in a movable aspect, according to the operation content, when the operation position is on the profile image.

### (Supplementary Description 3)

The image processing apparatus according to Supplementary Description 1 or 2,
wherein the internal structure image output unit is configured to display the internal structure image in an aspect that enables at least one of enlargement and reduction, according to the operation content of the pointing device accepted by the input accepting unit, when the operation position of the pointing device accepted by the input accepting unit is on the internal structure image.

### (Supplementary Description 4)

The image processing apparatus according to any one of Supplementary Descriptions 1 to 3,
wherein the input accepting unit is configured to accept the change in at least one of the first depth and the second depth, according to the operation content of the pointing device, when the operation position of the pointing device is on the first region of the profile image.

### (Supplementary Description 5)

The image processing apparatus according to Supplementary Description 4,
wherein the input accepting unit is configured to accept a change in the other one, other than the one described above, of the first depth and the second depth, according to the operation content of the pointing device, when the operation position of the pointing device is on the second region of the profile image.

### (Supplementary Description 6)

The image processing apparatus according to any one of Supplementary Descriptions 1 to 5,
wherein the input accepting unit is configured to accept the change to move the depth range in at least one of the deep direction and the shallow direction, according to the operation content of the pointing device, without changing the size of the depth range, when the operation position of the pointing device is on the third region of the profile image.

### (Supplementary Description 7)

The image processing apparatus according to any one of Supplementary Descriptions 1 to 6,
wherein the input accepting unit is configured to accept the change that enables at least one of enlargement and reduction of the depth range, according to the operation content of the pointing device, without changing the center of the depth range, when the operation position of the pointing device is on the profile image.

### (Supplementary Description 8)

The image processing apparatus according to any one of Supplementary Descriptions 1 to 7,
wherein the volume data is obtained by image reconstruction with signal data of the photoacoustic waves generated by light irradiation to a living body.

### (Supplementary Description 9)

An image processing method, including:
obtaining volume data indicating a three-dimensional internal structure of an object;
obtaining information on a first depth and a second depth from a predetermined reference surface, and color parameters of an internal structure image based on volume data in a depth range from the first depth to the second depth;
constructing the internal structure image in which the color parameters are allowed to vary continuously from the first depth to the second depth, based on the volume data and the color parameters, and displaying the internal structure image on a display device;
displaying on the display device a profile image indicating correspondence relation between a depth from the reference surface and the color parameter;
determining the input from the pointing device; and
determining the operation position of the pointing device.

### (Supplementary Description 10)

A non-transitory computer readable recording medium including a program recorded therein,
the program making a computer execute the following procedures:
   obtaining volume data indicating a three-dimensional internal structure of an object;
   obtaining information on a first depth and a second depth from a predetermined reference surface, and color parameters of an internal structure image based on the volume data in a depth range from the first depth to the second depth;
   constructing the internal structure image in which the color parameters are allowed to vary continuously from the first depth to the second depth, based on the volume data and the color parameters, and displaying the internal structure image on a display device;
   displaying on the display device a profile image indicating correspondence relation between a depth from the reference surface and the color parameter;
   determining the input from the pointing device; and
   determining an operation position of the pointing device.

### (Supplementary Description 11)

An image processing method, including:
making an internal structure image output unit construct an internal structure image, in which color parameters are allowed to vary continuously from a first depth to a second depth, in a depth range of the first depth or more and the second depth or less from a predetermined reference surface, based on volume data indicating a three-dimensional internal structure of an object, and display the internal structure image on a display device;
making a profile image output unit display on the display device a profile image indicating correspondence relation between a depth from the reference surface and the color parameter; and
making an input accepting unit accept an operation of a pointing device,
the image processing method, further including:
   making the input accepting unit accept change in at least one of the first depth and the second depth, according to the operation content of the pointing device, when the operation position of the pointing device is on the profile image,
   making the internal structure image output unit reconstruct the internal structure image, in which color parameters in the depth range after change accepted by the input accepting unit are allowed to vary, from the first depth to the second depth after change, similarly to a variation aspect of the color parameters before change, and redisplay the internal structure image on the display device, and
   making the profile image output unit reconstruct the profile image, according to the first depth and the second depth after change accepted by the input accepting unit, and redisplay the profile image on the display device.

### (Supplementary Description 12)

A non-transitory computer readable recording medium including a program recorded therein,
the program making a computer realize:
   an internal structure image output unit that constructs an internal structure image, in which color parameters are allowed to vary continuously from a first depth to a second depth, in a depth range of the first depth or more and the second depth or less from a predetermined reference surface, based on volume data indicating a three-dimensional internal structure of an object, and displays the internal structure image on a display device;
   a profile image output unit that displays on the display device a profile image indicating correspondence relation between a depth from the reference surface and the color parameter; and
   an input accepting unit that accepts operations of a pointing device,
wherein
   the input accepting unit is configured to accept a change in at least one of the first depth and the second depth, according to the operation content of the pointing device, when the operation position of the pointing device is on the profile image,
   the internal structure image output unit is configured to reconstruct the internal structure image, in which color parameters in the depth range after change accepted by the input accepting unit are allowed to vary, from the first depth to the second depth after change, similarly to a variation aspect of the color parameters before change, and to redisplay the internal structure image on the display device, and
   the profile image output unit is configured to reconstruct the profile image according to the first depth and the second depth after change accepted by the input accepting unit, and to redisplay the profile image on the display device.

### REFERENCE SIGNS LIST

10 Image processing apparatus
11 Internal structure image output unit
12 Profile image output unit
13 Input accepting unit
14 Pointing device
15 Slider image output unit
20 Display device
30 Internal structure image
40 Profile image
41 Color bar
42 Index
50 Pointer
60 First slider image
61 First slider bar
70 Second slider image
71 Second slider bar
100 Image processor
101 Data storage unit (SSD/HDD)
102 Central processing unit (CPU)
103 Graphics processing unit (GPU)
104 Video random access memory (VRAM)
105 Random access memory (RAM)
106 Communication unit
107 Volume data memory (region)
108 Internal structure image memory (region)
109 Profile image memory (region)
110 Network file server
111 Photoacoustic wave imaging device
S100 Volume data obtaining step
S110 Depth information and color parameter obtaining step
S120 Internal structure image displaying step
S130 Profile image displaying step
S140 Input determination step
S150 Operation position determination step
S160 Processing step

1.-10. (canceled)

11. An image processing apparatus, comprising:
an internal structure image output unit that constructs an internal structure image, in which color parameters are allowed to vary continuously from a first depth to a second depth, in a depth range of the first depth or more and the second depth or less from a predetermined reference surface, based on volume data indicating a three-dimensional internal structure of an object, and displays the internal structure image on a display device;
a profile image output unit that displays on the display device a profile image having a group of numerical values indicating a depth from the reference surface and an image in which the color parameters are allowed to vary, in order to indicate correspondence relation between the depth and the color parameter; and
an input accepting unit that accepts operations of a pointing device,
wherein
the input accepting unit is configured to accept the change of at least one of enlargement and reduction of the internal structure image, when an operation position of the pointing device is on the internal structure image, and is configured to accept a change in at least one of the first depth and the second depth, according to an operation content of the pointing device, when the operation position of the pointing device is on the profile image,
the internal structure image output unit is configured to display the internal structure image in an aspect that enables at least one of enlargement and reduction, according to the operation content of the pointing device accepted by the input accepting unit, when the operation position of the pointing device accepted by the input accepting unit is on the internal structure image, and is configured to reconstruct the internal structure image, in which color parameters in the depth range after change accepted by the input accepting unit are allowed to vary, from the first depth to the second depth after change, similarly to a variation aspect of the color parameters before change, and to redisplay the internal structure image on the display device, when the operation position of the pointing device accepted by the input accepting unit is on the profile image, and
the profile image output unit is configured to reconstruct the group of numerical values of the profile image, according to the first depth and the second depth after change accepted by the input accepting unit, and to redisplay the profile image on the display device.

12. An image processing apparatus, comprising:
an internal structure image output unit that constructs an internal structure image, in which color parameters are allowed to vary continuously from a first depth to a second depth, in a depth range of the first depth or more and the second depth or less from a predetermined reference surface, based on volume data indicating a three-dimensional internal structure of an object, and displays the internal structure image on a display device;
a profile image output unit that displays on the display device a profile image having a group of numerical values indicating a depth from the reference surface and an image in which the color parameters are allowed to vary, in order to indicate correspondence relation between the depth and the color parameter; and
an input accepting unit that accepts operations of a pointing device,
wherein
the input accepting unit is configured to accept the change in at least one in the first depth and the second depth, according to an operation content of the pointing device, when an operation position of the pointing device is on the first region of the profile image, and is configured to accept a change in the other one, other than the one described above, of the first depth and the second depth, according to the operation content of the pointing device, when the operation position of the pointing device is on the second region of the profile image,
the internal structure image output unit is configured to reconstruct the internal structure image, in which color parameters in the depth range after change accepted by the input accepting unit are allowed to vary from the first depth to the second depth after change, similarly to a variation aspect of the color parameters before change, and to redisplay the internal structure image on the display device, and
the profile image output unit is configured to reconstruct the group of numerical values of the profile image, according to the first depth and the second depth after change accepted by the input accepting unit, and to redisplay the profile image on the display device.

13. The image processing apparatus according to claim 11,
wherein the internal structure image output unit is configured to display the internal structure image in a movable aspect, according to the operation content of the pointing device accepted by the input accepting unit, when the operation position of the pointing device accepted by the input accepting unit is on the internal structure image, and
the profile image output unit is configured to display the profile image in a movable aspect, according to the operation content, when the operation position is on the profile image.

14. The image processing apparatus according to claim 12,
wherein the internal structure image output unit is configured to display the internal structure image in an aspect that enables at least one of enlargement and reduction, according to the operation content of the pointing device accepted by the input accepting unit, when the operation position of the pointing device accepted by the input accepting unit is on the internal structure image.

15. The image processing apparatus according to claim 12,
wherein the input accepting unit is configured to accept the change to move the depth range in at least one of the deep direction and the shallow direction, according to the operation content of the pointing device, without changing the size of the depth range, when the operation position of the pointing device is on the third region of the profile image.

16. The image processing apparatus according to claim 11,
wherein the input accepting unit is configured to accept the change that enables at least one of enlargement and reduction of the depth range, according to the operation content of the pointing device, without changing the center of the depth range, when the operation position of the pointing device is on the profile image.

17. The image processing apparatus according to claim 11,
wherein the profile image output unit is configured to reconstruct only the group of numerical values of the profile image, according to the first depth and the second depth after change accepted by the input accepting unit, without changing the image in which the color parameters are allowed to vary.

18. The image processing apparatus according to claim 11,
wherein the volume data is obtained by image reconstruction with signal data of the photoacoustic waves generated by light irradiation to a living body.

19. The image processing apparatus according to claim 11,
wherein the reference surface is a continuous surface that defines an interface of the object.

20. An image processing method, comprising:
making an internal structure image output unit construct an internal structure image, in which color parameters are allowed to vary continuously from a first depth to a second depth, in a depth range of the first depth or more and the second depth or less from a predetermined reference surface, based on volume data indicating a three-dimensional internal structure of an object, and display the internal structure image on a display device;
making a profile image output unit display on the display device a profile image having a group of numerical values indicating a depth from the reference surface and an image in which the color parameters are allowed to vary, in order to indicate correspondence relation between the depth and the color parameter; and
making an input accepting unit accept an operation of a pointing device,
the image processing method further comprising:
making the input accepting unit accept the change of at least one of enlargement and reduction of the internal structure image, when an operation position of the pointing device is on the internal structure image, and accept a change in at least one of the first depth and the second depth, according to an operation content of the pointing device, when the operation position of the pointing device is on the profile image,
making the internal structure image output unit display the internal structure image in an aspect that enables at least one of enlargement and reduction, according to the operation content of the pointing device accepted by the input accepting unit, when the operation position of the pointing device accepted by the input accepting unit is on the internal structure image, and reconstruct the internal structure image, in which color parameters in the depth range after change accepted by the input accepting unit are allowed to vary from the first depth to the second depth after change, similarly to a variation aspect of the color parameters before change, and redisplay the internal structure image on the display device, when the operation position of the pointing device accepted by the input accepting unit is on the profile image, and
making the profile image output unit reconstruct the group of numerical values of the profile image, according to the first depth and the second depth after change accepted by the input accepting unit, and redisplay the profile image on the display device.

21. An image processing method, comprising:
making an internal structure image output unit construct an internal structure image in, which color parameters are allowed to vary continuously from a first depth to a second depth, in a depth range of the first depth or more and the second depth or less from a predetermined reference surface, based on volume data indicating a three-dimensional internal structure of an object, and display the internal structure image on a display device;
making a profile image output unit display on the display device a profile image having a group of numerical values indicating a depth from the reference surface and an image, in which the color parameters are allowed to vary, in order to indicate correspondence relation between the depth and the color parameter; and
making an input accepting unit accept an operation of a pointing device,
the image processing method further comprising:
making the input accepting unit accept the change in at least one of the first depth and the second depth, according to an operation content of the pointing device, when an operation position of the pointing device is on the first region of the profile image, and accept a change in the other one, other than the one described above, of the first depth and the second depth, according to the operation content of the pointing device, when the operation position of the pointing device is on the second region of the profile image,
making the internal structure image output unit reconstruct the internal structure image, in which color parameters in the depth range after change accepted by the input accepting unit are allowed to vary from the first depth to the second depth after change, similarly to a variation aspect of the color parameters before change, and redisplay the internal structure image on the display device, and
making the profile image output unit reconstruct the group of numerical values of the profile image, according to the first depth and the second depth after change accepted by the input accepting unit, and redisplay the profile image on the display device.

22. A non-transitory computer readable recording medium comprising a program recorded therein which executes the image processing method according to claim 20.
